(19) United States
(12) Patent Application Publication
(10) Pub. No.: US 2011/0053239 A1
(43) Pub. Date: Mar. 3, 2011

(54) METHODS

(75) Inventors: Michael Jason Ray, London (GB); Richard James Murphy, London (GB)

(73) Assignee: IMPERIAL INNOVATIONS LIMITED, LONDON (GB)

(21) Appl. No.: 12/937,024

(22) PCT Filed: Apr. 9, 2009
(86) PCT No.: PCT/GB2009/000935

§ 371 Date: Nov. 12, 2010

(30) Foreign Application Priority Data
Apr. 11, 2008 (GB) 0806569.0

(51) Int. Cl. C12P 1/02

(52) U.S. Cl. 435/171

(57) ABSTRACT

A method of pre-treating lignocellulosic and cellulosic biomass for utilization of carbohydrate or lignin from the biomass, the method comprising the steps of inoculating the lignocellulosic biomass with lignocellulose degrading brown rot fungus and incubating the inoculated biomass under conditions in which growth of the lignocellulose degrading brown rot is promoted. The incubation may be under conditions in which growth of the lignocellulose degrading brown rot is promoted is terminated after less than 50 days or before substantial depletion of glucose in the biomass occurs.

The present invention relates to pre-treatment of cellulosic or lignocellulosic biomass, for example prior to saccharification, fermentation and ethanol recovery.

Lignocellulosic biomass is a term that refers to plant material composed of cellulose, lignin and hemicelluloses. These three cell wall components can be found in differing amounts depending on the plant species, nutrient availability and climatic conditions. Cellulose is the main structural component of plant cell walls and has a degree of polymerization that ranges from 500 to 20,000. Cellulose molecules are linear, unbranched β, 1-4 linked glucose polymers and have a strong tendency to form inter- and intra-molecular hydrogen bonds. Bundles of cellulose molecules aggregate to form microfibrils in which highly ordered (crystalline) regions alternate with less ordered (amorphous) regions. Microfibrils in turn make up fibrils and finally cellulose fibres. As a consequence of its fibrous structure and strong hydrogen bonds, cellulose has a very high tensile strength and is insoluble in most solvents.

Hemicellulose are heteropolysaccharides and are formed by a variety of monomers, that describe the non-cellulosic polysaccharide component of the plant cell wall. The most common monomers are glucose, galactose, rhamnose and mannose (the hexoses), xylose, fucose and arabinose (the pentoses) and can also include the uronic acids of glucose and galactose (Eaton and Hale, 1993b). Most hemicelluloses have a degree of polymerization of approximately 50-300 considerably less than cellulose. Hemicelluloses can be classified in three main families, xylans, mannans and galactans, named for the backbone polymer.

Lignin is a three dimensional macromolecule of very high molecular weight. Lignin is an amorphous and extensively cross-linked biopolymer. Lignin synthesis is via the polymerization of three monomeric phenylpropane units: Sinapyl, p-coumaryl and coniferyl alcohol (Boerjan et al., 2003). Lignin provides strength and rigidity by binding cellulose microfibrils together. It is hydrophobic in nature and influences the swelling characteristics of the plant cell wall, minimises water loss from the vascular system and can afford resistance to enzymatic degradation.

Cellulosic biomass is a term that refers to biomass that is composed principally of cellulose. Examples include paper, waste cotton textiles or cotton processing waste. Lignocellulose is defined in the UN FAO Glossary of biotechnology and genetic engineering as “The combination of lignin, hemicellulose and cellulose that forms the structural framework of plant cell walls.” (see FAO Research and Technology Paper No. 7, accessed 7 Apr. 2008 at http://www.fao.org/DOCREP/004/Y2775E/Y2775E00.HTM). There is a spectrum of composition from cellulosic biomass to lignocellulosic biomass related to the quantity of lignin (and consequently the proportions of cellulose, hemicelluloses and other components) comprising the biomass. This may range from very low levels (for example less than 1% lignin by mass) in ‘cellulosic’ biomass to relatively high levels (for example 30% lignin by mass as in wood) in lignocellulosic biomass. Typically lignocellulosic biomass may have a lignin content greater than about 4% i.e below about 4% lignin biomass is regarded as primarily ‘cellulosic’ in behavior and above about 7% its behavior is more in the lignocellulosic area.

Ethanol production from lignocellulosic biomass following saccharification (hydrolysis of the sugars) and fermentation is of considerable commercial interest. Extraction of lignin, other phenolic compounds, pectins, hemicelluloses components (and possibly other components) is also of value, as is saccharification in order to improve the value of the lignocellulosic biomass as animal feed (Pu at al., 2008; Ragauskas et al., 2006; Rogers et al., 2007; Sun et al., 2007; Wyman, 2002).

There are many existing kinds of pre-treatments which have been applied to biomass. The typical treatments are as follows:

Biological Pretreatments: Biological pretreatments employ fungi, typically white rot fungi, such as Trametes versicolor and Phanerochaete chrysosporium, for microbial de-lignification (Dorado et al., 2001; Gutierrez et al., 2001; Helmy and El-Meligi, 2002). However, fungal degradation can be a slow process and most fungi attack not only lignin, but also cellulose, thus resulting in the depletion of the overall sugar available for subsequent use.

Physical Pretreatments: Physical pretreatments can be classified in two general categories: mechanical (forms of milling) and non-mechanical (for example high-pressure steaming, high energy radiation and pyrolysis). During mechanical pretreatments, physical forces, (for example shearing or crushing) subdivide lignocellulose into finer particles. These physical forces can reduce cellulose crystallinity, particle size and degree of polymerization and increase bulk density. These structural changes result in a material that may be more accessible to subsequent treatments, but mechanical treatment is energy-intensive and operator time-intensive and therefore may not be practical on its own. Non-mechanical physical pretreatment methods can also increase digestibility, but have similar disadvantages.

Physicochemical Pretreatments: Steam explosion, Ammonia Fiber Explosion (AFEX) and sulphur dioxide catalysed steam explosion are examples of physicochemical pretreatments. In steam explosion wetted lignocellulose is heated to high temperatures (about 250° C.) and the pressure rapidly released, leading to particle size reduction. The high temperatures in combination with some chemical treatments can produce acetic acid from hemicellulose, so there is some autohydrolysis of the biomass (Nigam, 2002). These changes can result in better accessibility for subsequent treatments, but the severe conditions also produce degradation products that can inhibit hydrolysis and fermentation. These products can be removed by washing with water, but this also removes water soluble hemicellulose, which may be undesirable in some circumstances. Thus, there are disadvantages with these methods.

Chemical Pretreatments: Many chemical treatments have been used to alter the structure of the biomass cell wall to make the carbohydrate components more accessible for saccharification. Examples include the use of aqueous lime or sodium hydroxide, ammonia, dilute acid, oxidizing agents and solvent extraction agents. With all of these pretreatments the exact nature of the biomass has an impact on the overall efficiency of the process. Chemical pretreatments also have the potential to produce inhibitors that negatively affect the subsequent steps of saccharification and fermentation.

Accordingly, there is a need for alternative pretreatments.

The listing or discussion of an apparently prior-published document in this specification should not necessarily be taken as an acknowledgement that the document is part of the state of the art or is common general knowledge.

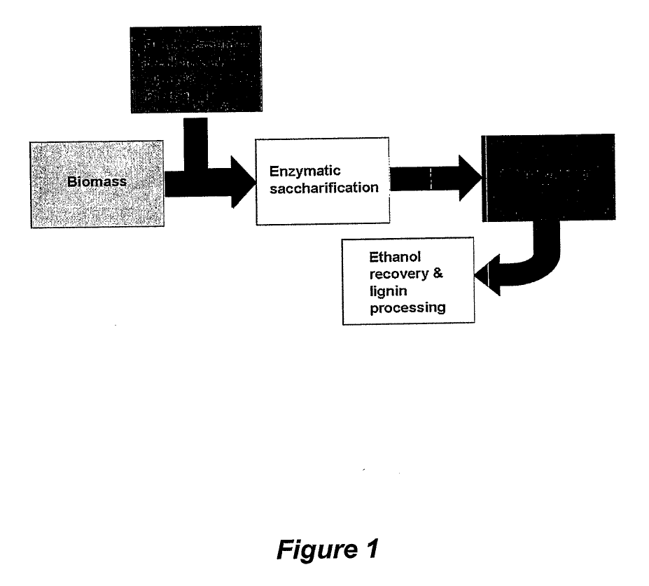

We have found that the mechanisms of Brown Rot (BR) wood decay fungi (previously unresearched in this context) can be put to use as biological ‘pre-treatments’ (see FIG. 1), for example providing enhanced saccharification and utilisation of carbohydrate and lignin from lignocellulosic or cellulosic feedstocks (e.g. softwoods, cereal straws, giant grasses, waste paper, waste cotton textiles or cotton processing waste) under controlled conditions (bulk biomass inoculation, saccharification, fermentation).

The term ‘brown rot’ is also used in the plant pathology field and refers to a type of rot on maturing or ripening fruit. The causal organisms of this type of rot belong to different fungal lineages and are distinct from the predominantly Basidiomycete fungi responsible for brown rot decay of wood and related lignocellulosic biomass that are the subject of this invention.

A first aspect of the invention provides the use of lignocellulose degrading brown rot fungi in a method of pre-treating lignocellulosic or cellulosic biomass for utilisation of carbohydrate or lignin from the biomass. For example, the lignin remaining following brown rot fungal pretreatment could be used in further processes for the production of economically significant chemicals or could be burned to produce energy for the ethanol production process (Pu et al., 2008; Ragauskas et al., 2006).

A second aspect of the invention provides a method of pre-treating lignocellulosic or cellulosic biomass for utilisation of carbohydrate or lignin from the biomass, the method comprising the steps of inoculating the lignocellulosic or cellulosic biomass with lignocellulose degrading brown rot fungi and incubating the inoculated biomass under conditions in which growth of the lignocellulose degrading brown rot fungi is promoted. Thus, the inoculated biomass may be incubated at a temperature of 2-45 or 50° C., preferably, 5-40° C. or 10-35° C., and a wood moisture content that is above the fibre saturation point but the cell lumen void space is not saturated. The relevant moisture content depends upon the biomass type: for example there will be a substantial difference between those ideal for tropical hardwood and cereal straw. Typically the relevant moisture content corresponding to the criteria indicated above is between 25 and 150% on an oven dry basis, for example approx 50% in a softwood. The moisture content and the availability of oxygen are both critical factors in the development of brown rot fungi which are all obligate aerobic organisms. The moisture content of the biomass can be controlled by natural air drying, artificial drying, addition of water e.g., by sprinkling or addition with a liquid fungal inoculum. Moisture content is usually monitored gravimetrically though some electrical conductance or resistance meters are available that can provide some insight up to moisture contents of about 40 to 50%—above this they become inaccurate.

Lignocellulose degrading Brown Rot fungi cause a rapid and extensive depolymerization of the cellulose and hemi-cellulose components of the plant cell wall as well as a limited, but significant, modification of the lignin component, usually via demethoxylation very early on in the brown rot decay process (Nilsson, 1988). The mechanism of action on the cellulose (and in the inventors' opinion, other cell wall polymers) is considered to be via hydroxyl radicals or equally potent metallo-oxygen species possibly generated by a Fenton system, oxalic acid has also been implicated in playing a significant role in cellulose depolymerization by brown rot fungi (Koenigs, 1974). It has been noted that even the smallest cellulases are too large to penetrate the pores of wood in the S3 and S2 layer of the cell wall even at advanced decay stages (Green and Highley, 1997; Srebotnik and Messner, 1991). For this reason it is hypothesized that the hyphae of brown rot lignocellulose degrading fungi release low molecular weight and highly diffusible agents to cause the observed early depolymerization of the cell wall polymers prior to a subsequent enzymatic attack (which leads to assimilation by the organism of the products of this enzymatic action). Hydrogen peroxide (or a related chemical) and its reaction with ferrous iron in the wood has been proposed as a possible mechanism by which transient free radicals that cause oxidative breakage in some of the glucose pyranose rings in the cellulose chain can be generated by brown rot fungi (Green and Highley, 1997). Such an agent would disrupt the microcrystalline structure enabling cellulases to then act at the interface of the S3 layer and lumen.

BRF reduce the pH of their immediate environment and this is thought to favour activity of some non-enzymatic systems hypothesized to be active as well as cellulolytic enzyme activity (Goodell, 2003a). The very quick depolymerization of the holocellulose components of the cell wall leads to a rapid decrease in the strength of the decayed wood (Eaton and Hale, 1993b).

In an embodiment, the incubation is performed such that essentially only the very early (depolymerization) phase of the Brown Rot fungal decay mechanism takes place, essentially without the assimilation phase taking place. By performing the incubation in this way it is considered that the accessibility of carbohydrate and lignin components is increased, without the amount of carbohydrate being reduced by subsequent assimilation by the brown rot fungi. Thus, for example, the incubation under conditions in which growth of the lignocellulose degrading brown rot is promoted may be terminated before substantial depletion of glucose in the biomass occurs, for example after less than 50, 40, 30, 25, 24, 23, 22, 21, 20, 19, 18, 17, 16, 15, 14, 13, 12, 11, 10, 9, 8, 7, 6, 5 or 4 days, preferably around 10 to 25 days at temperatures of around 10 to 25° C. and optimal moisture content. This is discussed further below.

The pre-treatment is considered to improve the accessibility of carbohydrate and also of lignin (e.g. as seen by improved alkali solubility). It is considered that at the early stages of brown rot attack, as used in the above embodiment, that lignin is rendered more accessible.

By lignocellulose degrading Brown Rot (BR) is meant rot which mainly assimilate the polysaccharide components of the plant cell wall through extensive depolymerization of the holocellulose (Nilsson, 1988). Wood exposed to brown rot becomes increasingly soluble in dilute solutions of sodium hydroxide (Nilsson, 1988). Brown rot decay is initiated in wood when the moisture content is above the fibre saturation point (approximately 30% moisture content on an oven dry basis), but the cell lumen void space is not saturated and the temperature should be between 5-40° C., preferably 10-35° C. (species dependent) (Goodell, 2003a).

Brown rot is typically caused by Basidiomycete fungi, but other fungi may also be able to exhibit the brown rot decay characteristics.

Brown rot fungi grow mainly within the plant cell lumina. Brown rot fungi appear to have a preference for degradation of softwood timbers though there are many examples of their activity on hardwood timbers and other lignocellulosic biomass.

A fungus can be readily identified as causing lignocellulose degradation of the brown rot type from the following characteristics which are recognised by those familiar with the art. Usually a member of the Basdiomycota

Capable of causing extensive mass loss in wood (typically in softwood but also other lignocellulosic materials) through consumption of the carbohydrate components of the biomass and leaving a lignin-like material as a residue at the completion of the degradation cycle (Goodell, 2003a; Green and Highley, 1997).

The micromorphology of the decay pattern exhibits an extensive depolymerization of the cell walls of the lignocellulosic material at distance from the fungal hyphae—the decay is not localised to the hyphae (Eaton and Hale, 1993b).

During brown rot degradation of wood a darkening of the colour occurs from pale brown/cream to a brown/dark brown coloration and, on drying, the wood samples break up in a characteristic cubical cracking pattern (Goodell, 2003a).

The use of combinations of observation, mass loss assessment, strength properties of decayed wood, chemical analysis and microscopy techniques in accordance with the above may be readily employed to determine the characterisation of an unknown fungus as a lignoceullulose (wood) degrading brown rot fungus.

Examples of lignocellulose degrading brown rot fungi include the following (Desch and Dinwoodie, 1996; Eaton and Hale, 1993b; Goodell, 2003a; Green and Highley, 1997; Nilsson, 1988):

Postia placenta, Gleophyllum trabeum, Gleophyllum separium, Lentinus lepideus, Coniophora puteana, Coniophora arida, Coniophora eremophila Tyromyces palustris, Serpula lacrymans, Daedalea quercina, Antrodia serialis, Antrodia sinuosa, Antrodia vaillantii, Antrodia xantha, Meruliporia incrassate, Paxillus panuoides, Amyloporia xantha, Piptoporus betulinus, Wolfiporia cocous.

The most commonly recognised in the wood decay context are: Postia placenta, Gleophyllum trabeum, Coniophora puteana, Serpula lacrymans

Preferred BR fungi for use in the present invention, particularly in relation to softwood, include Coniophora puteana and Postia placenta. Piptoporus betulinus is an example of a BR fungus that is considered to be useful with birch wood. Mixtures of BR fungi may be used, as will be well known to those skilled in the art.

There are several possible different ‘systems’ for providing inocula to wood chips or other forms of wood e.g. logs, discs etc, for example a slurry of fungal hyphae/spores in an aqueous medium (with or without additional nutrients) applied by spraying or pouring and stirring/mixing; an inoculum of solid/semi-solid ‘pellets’ (e.g. colonised wheat/barley etc grains for ‘dry-mixing’ with wood chips; a spore suspension in an aqueous medium); an inoculum of brown rot infected wood chips.

We consider that BR pre-treatment of biomass, for example softwood, can offer an advantage in a commercial setting. It is considered that BR pre-treatment can facilitate a high sugar release from softwood and other cellulosic or lignocellulosic biomass or alternatively can make other steps in the processing chain more cost/energy-effective. In a ‘combinatorial’ approach BR pre-treatment may be used in conjunction with other forms of pre-treatment e.g. grinding, steam explosion or acid hydrolysis of the biomass. It is considered that improvement in at least one of energy saving (and reduction in greenhouse gas emissions), process time reduction or increased ethanol production can arise as a direct result of the BR pre-treatment. Preferred softwoods are Pinus radiata and Pinus sylvestris.

Following the pre-treatment, the biomass is typically subjected to a saccharification process either using 1) enzymes or 2) dilute acid or 3) some other saccharification procedure. The sugar solution is then typically fermented to ethanol. A variant is simultaneous saccharification and fermentation where both steps happen in ‘one tank’. Wastes/residues are then separated.

BR pretreatment is considered to cause depolymerization of the lignocellulose or cellulosic biomass for very little expenditure of energy or materials and therefore has the potential to have considerable benefits with regards to the amount of energy and/or processing chemicals used ‘downstream’ in the saccharification processes, when compared with the current physical and chemical pretreatments used.

Enzymatic saccharification of cellulose has the potential benefits compared with dilute acid hydrolysis due to the less severe processing conditions. Currently the largest cost associated with ethanol produced by the enzymatic approach is the cost of the enzymes. Research by the US DOE is focused at improving the enzymes used and producing them in the large quantities that will be required and at a reduced price. The pre-treatment step in the saccharification is estimated to account for approximately 20% of the total process cost for ethanol production from lignocellulose, assuming the US DOE predictions that the price of enzymes will significantly reduce in price compared with their current value. This is second only to the cost of the biomass itself (approximately 35% of the costs). It is considered that the use of BR pre-treatment can reduce the cost of the subsequent saccharification stage.

The BR pre-treatment may be undertaken at ethanol production facilities as an integrated part of their operation or, alternatively, can be undertaken ‘in the field’ close to biomass harvesting sites. Within this latter, ‘distributed’ approach offers potential savings in transport energy via energy densification and moisture content reduction close to the biomass harvesting sites before its transport to ethanol plants). The use of BR “in the field” is not expected to cause any problems relating to “escape” of the BR organisms, as these organisms are generally and naturally present in the environment (though at levels too low and variable to be likely to be useful in the present invention).

The lignocellulosic or cellulosic biomass is preferably softwood, for example pine or spruce, but could refer to any cellulose containing plant biomass. This softwood biomass may be generated in many ways, for example as a specifically grown biomass crop, as residue from forestry operations, as by-product from timber processing operations, as used paper or packaging material or as arboricultural ‘waste’. The lignocellulosic or cellulosic biomass may alternatively be, for example, hardwoods, Miscanthus, bamboo, cereal straw, maize, rice or wheat waste, oil palm waste, sugar cane bagasse, waste packaging materials such as cardboard, any cellulosic waste, textiles and paper.

As noted above, the incubation under conditions in which growth of the lignocellulose degrading brown rot is promoted may be terminated after less than 50, 40, 30, 25, 24, 23, 22, 21, 20, 19, 18, 17, 16, 15, 14, 13, 12, 11, 10, 9, 8, 7, 6, 5 or 4 days, preferably around 10 to 25 days at temperatures of around 10 to 25° C. and optimal moisture content; or before substantial depletion of glucose in the biomass occurs. The incubation can be terminated by, for example, starting the next phase of the treatment, for example saccharification; or by lowering or raising the temperature, for example by using steam pipes or pipes carrying cold water through the pile of biomass; or by raising or (more typically) lowering the moisture content. The length of incubation required will depend on, for example, the temperature and humidity during the incubation, as well as the type of substrate, its physical form and the BR fungi present. The length of incubation required may be determined using a monitoring system for example Gel Permeation Chromatography to analyse the reduction in degree of polymerization of the biomass and mass loss of the biomass. Characteristic values (established by experimentation on specific biomass types) for these parameters that afford advantage in saccharification and/or lignin and other component release from the lignocellulosic or cellulosic biomass can be used for process monitoring. Alternative BR process monitoring parameters that correlate with desired saccharification or other outcomes are envisaged for example, chemical analysis, pH monitoring or spectroscopic analysis of sugars. It is envisaged that an operative can make use of charts or tables setting out expected decay progression for particular substrates and BR fungi under different conditions, coupled with assessment of the local conditions i.e. temperature, humidity, state of substrate etc to manage BR processes or by experience based on previous uses of such a monitoring system or empirical assessment of the previous results of subsequent procedures carried out on the pretreated biomass. Methods similar to those used in Example 1 may be used.

The BR pre-treatment can be carried out to advantage on relatively large wood chips (circa. 10×5×30 mm). This would reduce the amount of mechanical processing work the biomass would require and consequently the amount of energy required. We consider that the Brown Rot pretreatment can be used on wood chips across quite a range and may also be applied to ‘whole’ wood e.g. sawlog offcuts, pulpwood ‘bolts’, etc. The Brown Rot pre-treatment can also be applied to other sorts of cellulosic and lignocellulosic biomass e.g. cereal straw, bamboo, Miscanthus, paper ‘waste’ etc—these will all have different optimal sixes etc for the treatment and some may require no size reduction.

As an example, the desired ‘early’ phase of BR may be up to when mass loss (in pine sapwood) is less than 5, 10 or 15% of the oven dry mass of the material. The graphs in Example 1 show that at 15 days, mass loss is about 7% and running at a linear rate from here and at the time glucose release in the saccharification assay is at a maximum (‘plateau’) level. Beyond this time (under our selected conditions) mass loss increases but glucose yield starts to decline. These two parameters mass loss and glucose yield can be used as measures that the BR pre-treatment process has been implemented to its optimum level.

Any published documents referred to herein are hereby incorporated by reference.

The invention is now illustrated further by reference to the following, non-limiting, Figures and Examples.

FIG. 1: Biomass to ethanol processing stages

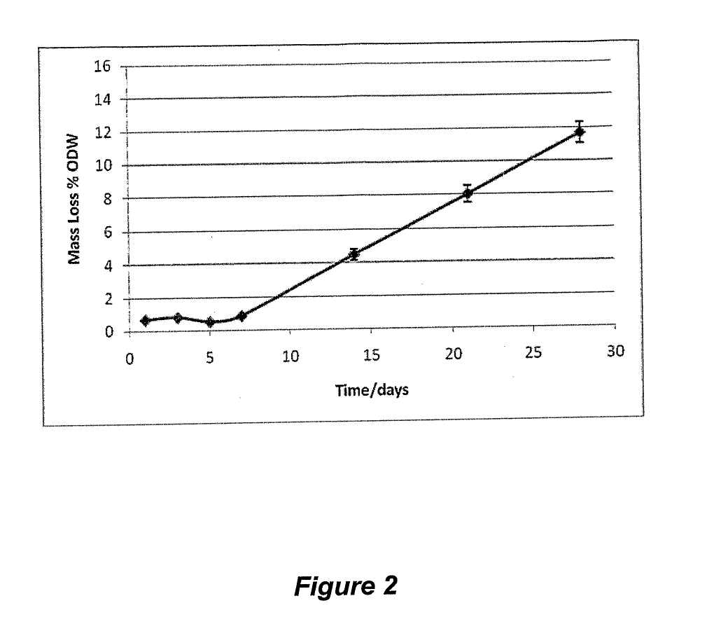

FIG. 2: Pinus sylvestris mass loss based on oven dry weight from sapwood blocks exposed to the brown rot fungus Coniophora puteana. All results are shown as means±standard error.

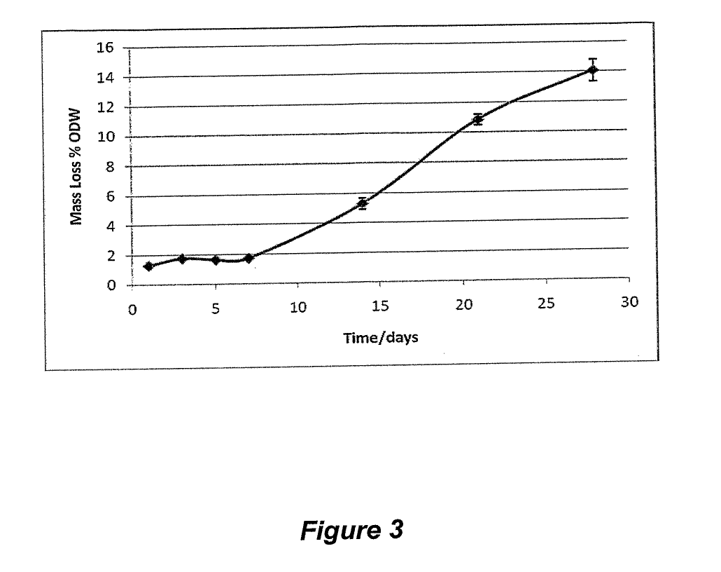

FIG. 3: Pinus sylvestris mass loss based on oven dry weight from sapwood blocks exposed to the brown rot fungus Postia placenta. All results are shown as means±standard error.

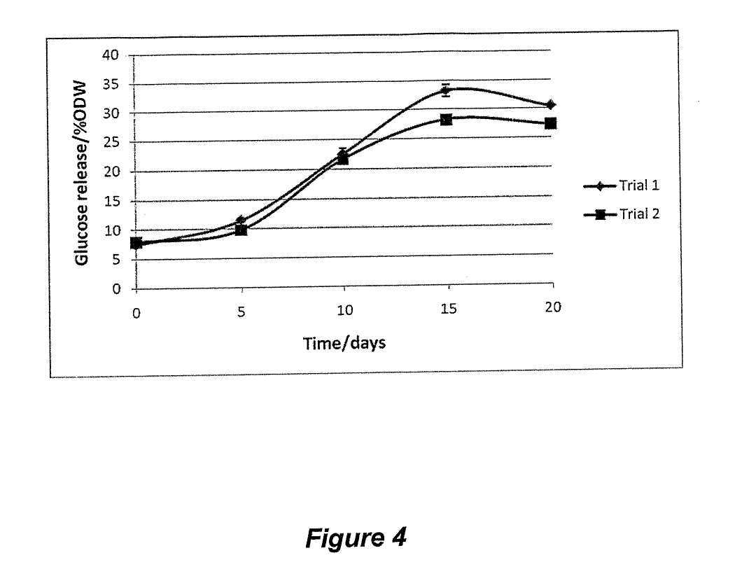

FIG. 4: Two independent trials of glucose yielded from the saccharification assay as a percentage of the Oven Dry Weight (ODW) of the Pinus sylvestris sapwood after exposure to the brown rot fungus Coniophora puteana. All results are shown as means±standard error.

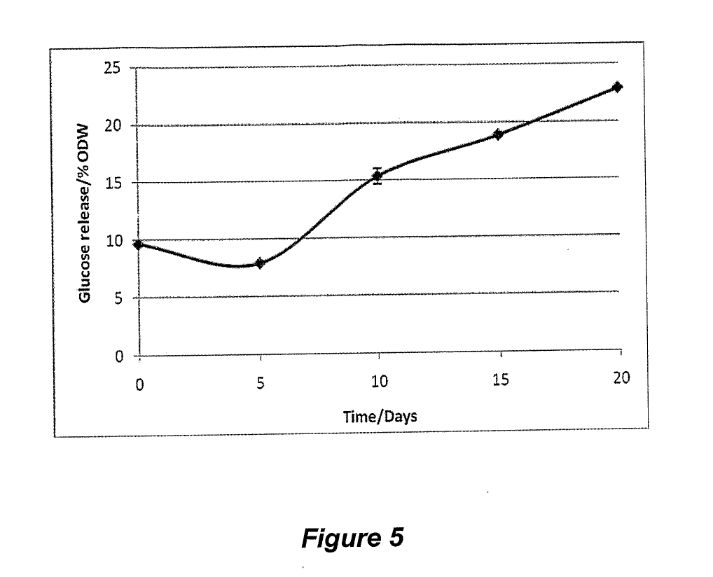

FIG. 5: Glucose yielded from the saccharification assay as a percentage of the Oven Dry Weight (ODW) of the Pinus sylvestris sapwood after exposure to the brown rot fungus Postia placenta. All results are shown as means±standard error.

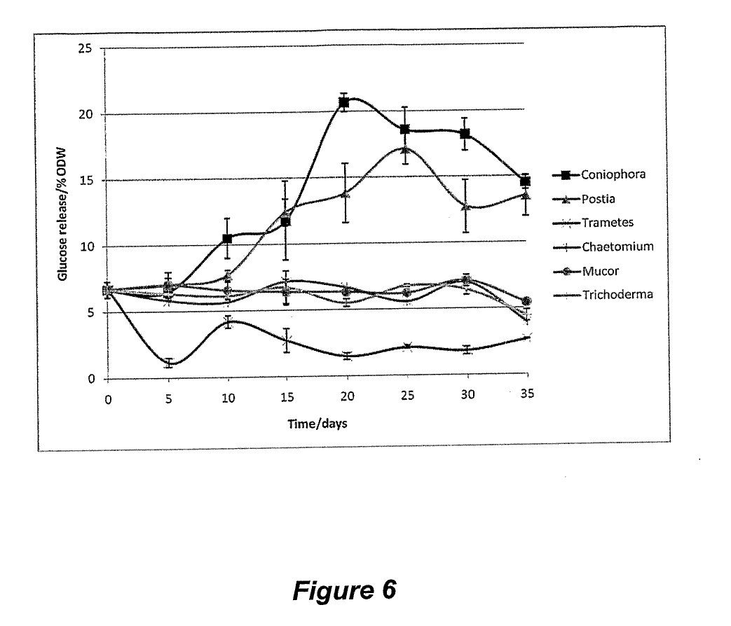

FIG. 6: Glucose yields from P. radiata sapwood blocks after exposure to a range of wood colonizing fungi. Released glucose is shown as a percentage of total Oven Dry Weight (ODW) of the biomass. n=3 (each replicate composed of eight homogenised wood blocks). All results are shown as means±standard error.

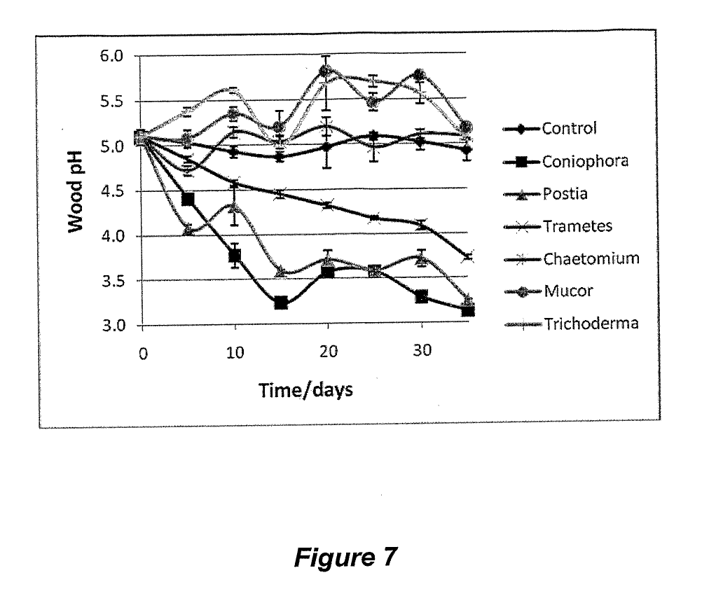

FIG. 7: Wood pH of the exposed Pinus radiata sapwood was determined from ground biomass material. n=3 (each replicate is composed of eight homogenised wood blocks). All results are shown as means±standard error.

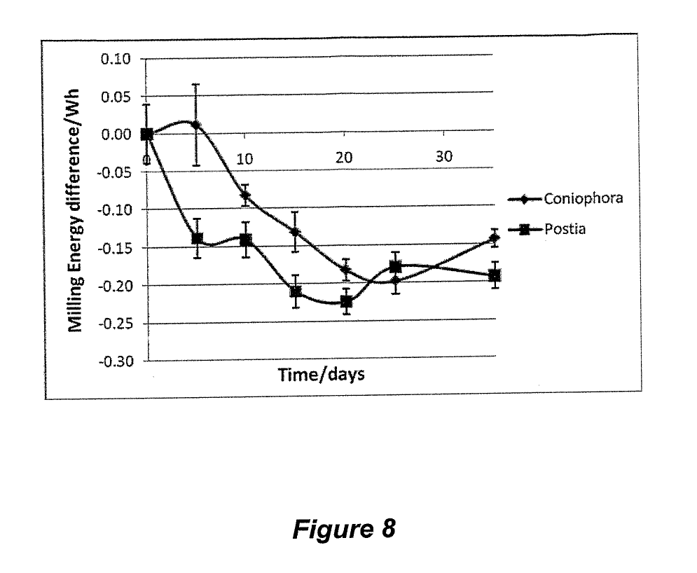

FIG. 8: Difference in energy required to mill 8 Pinus radiata mini-blocks after exposure to two brown rot fungi, when compared with non-exposed blocks. All results are shown as means±standard error.

### EXAMPLE 1

### The Optimisation of Sugar Yields Using Brown Rot Fungi as a Pre-Treatment Before Saccharification of Softwood (Pinus sylvestris) Biomass

Here we examined the degree of degradation over time of Scots pine (Pinus sylvestris) sapwood caused by BR fungi and the effects of early stages in the brown rot decay process on the yields of glucose from such pine wood. Two typical BR fungi were selected for analysis: Postia placenta and Coniophora puteana. These fungi were used in pure culture fungal decay tests to determine the extent of loss in oven dry mass of the wood as a measure of the degree of degradation.

Pine sapwood blocks (measuring approximately 5×30×10 mm) were transversely cut from air dried pine logs of approximately 150 mm diameter. The blocks were labelled, weighed and sterilised by gamma irradiation.

Pure fungal cultures of both Postia placenta and Coniophora puteana were used in this example and were cultured on 2% malt agar Petri dishes. Fungal cultures were grown for 10-14 days, or until the mycelial growth was near the edge of the Petri dish, prior to inoculation.

Sterilised Pine sapwood blocks were added to the fungal Petri dishes by placing stainless steel washers first on top of the mycelium followed by the pine sapwood blocks. These were incubated in a controlled temperature room at 25° C. with a RH (relative humidity) of 75% for up to 3 weeks.

Following harvest of the pine sapwood blocks from the Petri dishes some of the blocks were weighed, oven dried and re-weighed, to determine mass loss. The remainder of the blocks were ground and the material put through a saccharification assay following standard NREL laboratory protocols (Brown and Torget, 1996; Hames et al., 2005). Sugar analysis was carried out on a Jasco systems HPLC running an BioRad Aminex HPX-87P column.

Mass loss data presented in FIG. 2 and FIG. 3 show the relatively low mass losses i.e. approximately 5% after 15 days. The mass loss from the pine sapwood appears to be linear after about 7 days exposure.

FIG. 4 and FIG. 5 show the quantities of glucose released from samples of Pinus sylvestris sapwood exposed to the brown rot fungi Coniophora puteana and Postia placenta for various time periods and are given as a proportion of the total oven dry weight (ODW) of the biomass. These values are total glucose values and account for any glucose that may be present in the form of the hemicellulose component of the wood. This example indicates that a BR pre-treatment of pine sapwood can provide an almost four-fold increase in the amount of glucose released (FIG. 4 Error! Reference source not found.) representing a significant improvement from the glucose released from non-pre-treated pine wood. This compares favourably to the physicochemical pretreatment technologies currently employed (Ewanick et al., 2007; Frederick et al., 2008).

### EXAMPLE 2

### Brown Rot Fungi are Unique in Offering Improved Glucose Saccharification Yields from Softwood (Pinus Radiata) Biomass after Pretreatment

Here we show that after restricted exposure of pine sapwood to brown rot fungi, glucose yields following enzymatic saccharification are significantly increased. The results demonstrate the potential of using brown rot fungi as a biological pretreatment for biofuel production and we show that this will greatly reduce energy and chemical inputs for releasing fermentable carbohydrate compared to current pretreatment technologies.

To establish if the observed increase in glucose yields was specific to the actions of BR fungi, we investigated the effects of exposing a different softwood biomass (Pinus radiata) to six different fungi, all of which are known to colonize wood: Coniophora puteana (brown rot), Postia placenta (brown rot), Trametes versicolor (white rot), Chaetomium globosum (soft rot), Trichoderma viride (mould) and a species of Mucor (mould). The two mould fungi were used to represent organisms that can grow in wood but do not actively degrade it as a substrate. The other fungi will actively degrade wood under appropriate conditions.

Fungal exposure of wood was conducted by cutting transverse air dried wood blocks measuring 5×10×25 mm from Pinus radiata sapwood and sterilizing by gamma irradiation. Fungal cultures were grown on 4% malt agar for no more than 28 days prior to inoculation. Sterilised wood blocks were placed aseptically onto solid media on top of sterile stainless steel rings, to avoid direct contact with the agar. Fungi and wood blocks were incubated in a controlled temperature room at 22° C. for up to 35 days.

The saccharification assay followed a modified protocol based on Selig et al. (Selig et al., 2008), the differences being 60 FPU/g oven dry biomass of cellulase (Celluclast 1.5 L, Sigma, UK) and 64 pNPGU/g oven dry biomass of β-glucosidase (Novozyme 188, Sigma, UK). Assays were incubated at 50° C. for 168 hours. Released glucose was analysed by HPLC on an Agilent 1200 series HPLC fitted with a BioRad aminex HPX-87P column with a H₂O mobile phase.

Alongside the saccharification of the wood the pH of the wood was also measured. The biomass was milled and sieved to a defined particle size between 180-850 μm. 100 mg ODW of this was added to boiling deionised water and incubated for 20 minutes. Following this, samples were removed and allowed to cool before pH was measured with a WTW Inolab pH meter.

The results in FIG. 6 show that treatment with the two BR fungi enhances the glucose released (approximately 3-fold) following enzymatic saccharification. Exposure of the wood to the mould and soft rot fungi had no effect on glucose release by enzymatic saccharification.

FIG. 7 shows a steep decline from pH 5 to approximately pH 3 to 3.5 of the wood after exposure to the two BR fungi C. puteana and P. placenta when compared with the unexposed controls and the non wood decay-fungi. T. versicolor also showed a decline, but at a slower rate than the BR fungi. This is consistent with the literature reports regarding the differences between BR and white rot fungi and their distinctive decay mechanisms (Goodell, 2003b). BR fungi produce oxalic acid and it is suggested that this stimulates the non-enzymatic depolymerization observed in BR decayed wood via a Fenton reaction which is known to generate hydroxyl radicals (by a reaction of H₂O₂ and Fe(II))(Espejo and Agosin, 1991). It is our hypothesis that this depolymerization of the cellulose molecules is partially or wholly responsible for the observed increase in the glucose saccharification yields after exposure to BR fungi. The depolymerization of the carbohydrates in the cell wall causes a marked reduction in strength of the wood (Eaton and Hale, 1993a) and we have also observed concomitant reductions in the energy required to grind BR exposed wood compared with unexposed wood (FIG. 8).

Because BR pretreatment uses mild conditions a further advantage for lignocellulosic ethanol production should be in the absence/reduced level of fermentation inhibitors commonly generated with more severe pretreatments. We assessed this by performing ethanol fermentations with Saccharomyces cerevisiae on glucose solutions generated by the enzymatic saccharification of P. radiata wood pretreated by exposure to C. puteana (20 days) or P. placenta (25 days).

All fermentations were carried out at 0.5% (w/v) glucose concentration with 1% (w/v) yeast extract and 2% (w/v) peptone in a final volume of 15 mL and incubated at 30° C. for 24 hours. Ethanol and glucose were analysed by HPLC on a Jasco Systems HPLC fitted with a BioRad aminex HPX-87H column. The fermentation data was assessed using an unpaired two-tailed Student's t-test. The null hypothesis stated there was no significant difference between the means. The null hypothesis was rejected when P<0.05.

No significant difference in the rate and yield of ethanol production was observed when comparing sugar solutions from BR pretreated pine with controls of glucose alone (ethanol/glucose=0.42), indicating that fermentation inhibitors were not generated by the BR pretreatment.

### EXAMPLE 3

### Further Optimisation of the Brown Rot Fungi Treatment

Further optimisation of the BR treatment protocols can be achieved by the following methods:

‘Combinatorial’ approach—Coupling of BR pre-treatment to enhance the efficiency of other recognised pre-treatment methods (e.g. steam explosion, dilute acid).

Particle size—the BR pre-treatment can be carried out to advantage on relatively large wood chips (circa. 10×5×30 mm). This would reduce the amount of mechanical processing work the biomass would require and consequently the amount of energy required. We consider that the Brown Rot pretreatment can be used on wood chips across quite a range and may also be applied to ‘whole’ wood e.g. sawlog offcuts, pulpwood ‘bolts’, etc. The Brown Rot pre-treatment can also be applied to other sorts of cellulosic and lignocellulosic biomass e.g. cereal straw, bamboo, Miscanthus, paper ‘waste’ etc—these will all have different optimal sixes etc for the treatment and some may require no size reduction.

### REFERENCES

Boerjan, W., et al., 2003. Lignin biosynthesis. Annual Review of Plant Biology. 54, 519-546.
Brown, L., Torget, R., 1996. Enzymatic saccharification of Lignocellulosic Biomass. National Renewable Energy Laboratory. Laboratory Analytical Procedure 009.
Desch, H. E., Dinwoodie, J. M., 1996. Timber, structure, properties, conversion and use. Macmillan Press Ltd., London.
Dorado, J., et al., 2001. Utilization of white-rot fungi for pitch control in pulp and paper manufacturing. Afinidad. 58, 175-180.
Eaton, R. A., Hale, M. D. C., 1993a. Wood: decay, pests, and protection. Chapman & Hall, London.
Eaton, R. A., Hale, M. D. C., 1993b. Wood: Decay, pests and protection. Chapman and Hall, London.
Espejo, E., Agosin, E., 1991. Production and Degradation of Oxalic-Acid by Brown Rot Fungi. Applied and Environmental Microbiology. 57, 1980-1986.
Ewanick, S. M., et al., 2007. Acid-catalyzed steam pretreatment of lodgepole pine and subsequent enzymatic hydrolysis and fermentation to ethanol. Biotechnology and Bioengineering. 98, 737-746.
Frederick, W. J., et al., 2008. Production of ethanol from carbohydrates from loblolly pine: A technical and economic assessment. Bioresource Technology. 99, 5051-5057.
Goodell, B., Brown-rot fungal degradation of wood: our evolving view. In: B. Goodell, et al., Eds.), Wood deterioration and preservation. American Chemical Society, Washington D.C., 2003a, pp. 97-118.
Goodell, B., 2003b. Brown-rot fungal degradation of wood: Our evolving view. Wood Deterioration and Preservation. 845, 97-118.
Green, F., Highley, T. L., 1997. Mechanism of brown-rot decay: Paradigm or paradox. International Biodeterioration & Biodegradation. 39, 113-124.
Gutierrez, A., et al., 2001. The biotechnological control of pitch in paper pulp manufacturing. Trends in Biotechnology. 19, 340-348.
Hames, B., et al., 2005. Preparation of Samples for Compositional Analysis. National Renewable Energy Laboratory. Laboratory Analytical Procedure.
Helmy, S. M., El-Meligi, M., 2002. Biopulping and biobleaching by white rot fungi. Journal of Scientific & Industrial Research. 61, 376-381.
Koenigs, J. W., 1974: Production of Hydrogen-Peroxide by Wood-Rotting Fungi in Wood and Its Correlation with Weight-Loss, Depolymerization, and Ph Changes. Archives of Microbiology. 99, 129-145.
Nigam, J. N., 2002. Bioconversion of water-hyacinth (Eichhornia crassipes) hemicellulose acid hydrolysate to motor fuel ethanol by xylose-fermenting yeast. Journal of Biotechnology. 97, 107-116.
Nilsson, T., Defining fungal decay types: Final proposal., The international research group on wood preservation. IRG Secretariat, Sweden. IRG Doc. No. 88-1355, Madrid, Spain, 1988.
Pu, Y., et al., 2008. The new forestry biofuels sector. Biofuels, Bioproducts & Biorefining. 2, 58-73.
Ragauskas, A. J., et al., 2006. The path forward for biofuels and biomaterials. Science. 311, 484-489.
Rogers, P. L., et al., 2007. Zymomonas mobilis for fuel ethanol and higher value products. Biofuels. 108, 263-288.
Selig, M., et al., Enzymatic Saccharification of Lignocellulosic Biomass. National Renewable Energy Laboratory, 2008.
Srebotnik, E., Messner, K., 1991. Immunoelectron Microscopic Study of the Porosity of Brown-Rot Degraded Pine Wood. Holzforschung. 45, 95-101.
Sun, J. S., et al., 2007. Pretreatment technology of corn stover for ethanol production. Progress in Chemistry. 19, 1122-1128.
Wyman, C. E., 2002. Research and development needs for a fully sustainable biocommodity industry. Advancing Sustainability through Green Chemistry and Engineering. 823, 31-46.

1. (canceled)

2. A method of pre-treating lignocellulosic and cellulosic biomass for utilization of carbohydrate or lignin from the biomass, the method comprising the steps of inoculating the lignocellulosic biomass with lignocellulose degrading brown rot fungus and incubating the inoculated biomass under conditions in which growth of the lignocellulose degrading brown rot is promoted, wherein said incubation is terminated after less than 50, 40, 30, 25, 24, 23, 22, 21, 20, 19, 18, 17, 16, 15, 14, 13, 12, 11, 10, 9, 8, 7, 6, 5 or 4 days, or before substantial depletion of glucose in the biomass occurs, and wherein the pre-treatment is pre-treatment for any form of cellulose or hemicellulose hydrolysis/saccharification, or for another form of pre-treatment.

3. (canceled)

4. The method of claim 2 wherein said another form of pre-treatment is dilute acid hydrolysis, steam explosion or Ammonia Fiber Explosion.

5. The method of claim 2, wherein the pre-treatment is pre-treatment for enzymatic saccharification, fermentation and ethanol recovery or processing and/or lignin processing.

6. The method of claim 2, wherein the lignocellulosic biomass is softwood.

7. The method of claim 6 wherein the softwood is pine sapwood.

8. The method of claim 6 wherein the softwood is from the species Pinus radiata or Pinus sylvestris.

9. The method of claim 2, wherein the lignocellulose degrading brown rot fungus is or comprises Coniophora puteana or Postia placenta.

10. The method of claim 6 wherein the softwood is in the form of wood chips of average volume of 0.1-1 cm³.

11. The method of claim 10, wherein said average volume is 0.2-0.5 cm³.
